(19) United States
(12) Patent Application Publication
(10) Pub. No.: US 2009/0249513 A1
(43) Pub. Date: Oct. 1, 2009

(54) Method for Expression and Accumulation of Peptide in Plant

(75) Inventors: Tsukasa Matsuda, Aichi (JP); Naohito Aoki, Aichi (JP); Fujio Hashizume, Aichi (JP); Shingo Hino, Aichi (JP); Misako Kakehashi, Aichi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NATIONAL UNIVERSITY CORPORATION NAGOYA UNIVERSITY, Nagoya-shi (JP)

(21) Appl. No.: 11/885,762

(22) PCT Filed: Mar. 1, 2006
(86) PCT No.: PCT/JP2006/304421

§ 371 Date: Sep. 6, 2007

(30) Foreign Application Priority Data
Mar. 7, 2005 (JP) 2005-062996

(51) Int. Cl. A01H 1/00; C12N 15/63; A01H 5/00

(52) U.S. Cl. 800/278; 435/320.1; 800/298; 800/320.2

(57) ABSTRACT

The present invention relates to a method for expressing and accumulating a low-molecular peptide in plant seeds; a vector therefore; and a plant transformed with the vector. In the present invention, an intended peptide composed of 3 to 40 amino acid residues is expressed and accumulated in seeds of a plant by transforming the plant with a fusion protein expression vector comprising a gene encoding a member of the glutelin multigene family and two or more copies of a gene encoding the intended peptide ligated downstream of the gene under the control of a promoter.

### TECHNICAL FIELD

The present invention relates to a method for stably and abundantly expressing and accumulating a low-molecular peptide in a plant, particularly in plant seeds, to a vector therefor, and to a plant transformed with the vector.

### BACKGROUND ART

At present when food-born diseases such as cardiac disease, hypertension and allergy are increasing, a need exists for the supply of high-quality protein excellent in functionality. For this issue, there have been attempted the search for peptides having physiological functions useful in maintaining and improving health, and the design for high activation thereof. In addition, it has been attempted to develop crops in which proteins or peptides having such physiological functions are highly accumulated (see Patent Document 1).

However, when peptides produced employing Escherichia coli, yeast or animal cells, or recombinant plants are used as foods or medicines, it generally poses a problem of high cost involved in ensuring the safety thereof and in the large-scale culture, purification, and the like.

What is pointed out regarding the safety of recombinant crops is that these crops each have an antibiotic resistance gene, a selection marker, left. For a hygromycin resistance gene most widely used for the recombination of rice plant, for example, data on the safety thereof have not sufficiently been accumulated. A kanamycin resistance gene has been subjected to sufficient safety evaluation, but the safety thereof is still seen as a problem.

The abundant expression of an intended protein using a recombinant plant has previously been subjected to devices such as selecting a highly active promoter and stabilizing a translation product; however, the number of copies of an intended gene introduced into a host plant genome is also an important factor determining the expression level of the intended protein. Here, when the protein is transiently expressed using a redifferentiated plant or the like for research, multiple copies of the intended gene being dispersed on the plant genome is not a problem. In the case of a commercial recombinant plant, however, the dispersion of multiple copies of the intended gene on the plant genome makes it difficult to select and maintain a high expression (multicopy) line genetically stable over generations. In other words, the ability to collectively introduce multiple copies of gene into a narrow region (a single gene locus) of the host genome will lead to easily achieving a recombinant plant line which is genetically stable and has multiple copies of the intended gene. However, conventional techniques make random the location of the introduced genes on a host genome and render difficult the artificial control thereof; thus, it has not been easy to obtain a high-expression line with multiple copies of the intended gene (a high expression multicopy line).

Use of a plant as a host generally results in the easy decomposition of a low molecular weight compound such as a peptide in the seed, which makes difficult the stable and abundant expression and accumulation thereof. In fact, there is a report in which a low-molecular peptide (AMY, 29 amino acid residues) has been expressed as a fusion protein with ubiquitin in a tobacco plant (see non-Patent Document 1); however, the expression and accumulation of the peptide in the seed are not mentioned.

On the subject of the above-described selection marker, one of the present inventors has developed and reported a method for efficiently producing a plant which has a desired trait and from which a selection marker such as an antibiotic resistance gene is removed (see Patent Document 2). However, a sufficient solution has not yet been described to the problem of the stable expression and accumulation of a low-molecular peptide in a plant, particularly in plant seeds.

Patent Document 1: JP Patent Publication (Kokai) No. 2004-321079A

Patent Document 2: JP Patent Publication (Kokai) No. 2003-000082A

Non-Patent Document 1: Hondred D., et al., Plant Physiol. 1999 February; 119(2): 713-24.

### DISCLOSURE OF THE INVENTION

An object of the present invention is to develop a method for efficiently and abundantly expressing and accumulating a low-molecular peptide in a plant and provide a new recombinant crop having the enhanced physiological functionality.

To solve the above problems, the present inventors have designed an artificial synthetic gene deduced from an intended peptide sequence using codons most frequently found in a plant to be transformed and have allowed the gene to fuse alone or in a tandem repeats of plural molecules thereof with a seed storage protein gene. The fusion gene has been introduced into a rice plant by ligating downstream of a seed-specific promoter to abundantly express the gene as a fusion protein in the seeds. As a result, it has been demonstrated that a line of the plant highly expressing and accumulating the peptide is more frequently found in the case of introducing the plural copies of the gene than in the case of introducing a single copy of the gene.

Specifically, the present invention provides a fusion protein expression vector comprising a gene encoding a member of the glutelin multigene family and two or more copies of a gene encoding an intended peptide composed of 3 to 40 amino acid residues (hereinafter referred to as “intended gene”) ligated downstream of the gene encoding the member of the glutelin multigene family, under the control of a promoter.

Examples of the glutelin multigene family member can include glutelin A and glutelin B; an embodiment using glutelin B is herein described as a particularly preferred example.

The promoter is not particularly limited provided that it can function in a plant; however, preferred is the glutelin promoter having a strong promoter activity (for example, GluB-1 promoter or GluPF2 promoter) or the like.

The intended peptide is a low-molecular peptide composed of the order of 3 to 40 amino acid residues, preferably 3 to 30 amino acid residues, more preferably 3 to 20 amino acid residues. The vector of the present invention comprises two or more, preferably about 2 to 20 tandemly-ligated copies of a gene encoding the low-molecular peptide, and expresses a fusion protein of the glutelin multigene family member and the intended peptide.

The vector of the present invention comprising two or more ligated copies of an intended gene provides a high expression line with higher probability than a vector comprising a single copy of the gene. This is attributed to that the vector multiply infects a single gene locus to multiply introduce two or more ligated copies of the intended gene into the single gene locus.

The vector of the present invention is preferably designed so that the peptide ligation sites (the site between the glutelin multigene family member and the intended peptide and the site between molecules of the intended peptide) in an expressed fusion protein are each tyrosine or phenylalanine; this allows each constituent peptide molecule to be rapidly released from the fusion protein exposed to digestive enzyme activity.

As a preferred example of the intended peptide contained in the vector of the present invention, an epitope peptide of type II collagen can be exemplified.

As a preferred form of the vector of the present invention, a binary-type hybrid vector containing two T-DNA regions can be exemplified. The hybrid vector contains two or more copies of a gene encoding an intended peptide composed of 3 to 40 amino acid residues ligated to the gene encoding the member of the glutelin multigene family in the first T-DNA region and a selection marker in the second T-DNA region.

The present invention also provides a recombinant plant transformed with the vector of the present invention, cells, tissues, organs and seeds of the plant, and cultures thereof. As a preferred example of the plant, a rice plant can be exemplified. Particularly, the vector of the present invention can also be suitably used in the useful rice variety “Koshihikari” which is difficult to transform and has a high commercial value.

The present invention also provides a method for expressing and accumulating an intended peptide in a plant, particularly in plant seeds, comprising transforming the plant with the vector of the present invention and expressing the vector in seeds of the plant. The method may further comprise the step of selecting, by DNA analysis, a plant free of selection marker from selfed progenies of the transformed plant.

The present invention also provides a method for breeding a multicopy line containing the two or more copies of an intended gene multiply introduced into a single gene locus thereof by transforming a plant with the vector of the present invention.

According to the present invention, multiple copies of a gene can be introduced into a narrow region (a single gene locus) of the host genome, which enables the easy breeding of a high expression line which is genetically stable and has multiple copies of an intended gene. This makes it possible to safely, stably and inexpensively produce, in plant seeds, large amounts of a low-molecular peptide intended to be orally administered to humans, such as a tolerogenic peptide. The continuous oral ingestion thereof as a part of food enables the functionality (tolerogenicity or the like) of the peptide to be maximally exerted.

### BRIEF DESCRIPTION OF THE DRAWINGS

FIG. 1 is the construction scheme for a vector for transforming a rice plant, containing GluA cDNA and HuCII cDNA;

FIG. 2 is the structure of a vector for yeast secretory expression containing GluA cDNA and HuCII cDNA;

FIG. 3 is an electrophoretic photograph showing the results of the affinity purification of an anti-HuCII antibody in yeast (A) and the results of the Western blot analysis of HuCII in the yeast culture supernatant using the antibody;

FIG. 4 is the structure of vector pSB426Glu-C4;

FIG. 5 is a set of photographs of Western analysis patterns showing the results of detecting GluA-HuCII fusion proteins in T1 seeds (for reference: Glutelin: 54 kDa (33 kDa+21 kDa), [HuCII]×1: 3 kDa, ×4: 11 kDa and ×8: 22 kDa);

FIG. 6 is the results of half-seed analysis (A: the PCR analysis of seedling leaf DNA, B: the Western analysis thereof);

FIG. 7 is the prevalence of a line free of selection marker (upper: [HuCII]×8, middle: [HuCII]×4, and lower: [HuCII]×1);

FIG. 8 is the accumulation of a GluA-HuCII fusion protein in the protein body (high density fraction);

FIG. 9 is the analysis of proteins in T1 fixed lines free of selection marker;

FIG. 10 is the Southern blot analysis in T1, T2 and T3 fixed lines which show extremely high expression because an intended gene is multiply inserted although they contain a selection marker. The multiply inserted intended gene is stably inherited for three generations without being deleted; and

FIG. 11 is the evaluation of the suppressive effect of ingestion of HuCII-containing TG rice on collagen immunogenicity. “A” indicates a group having ingested a feed containing the TG rice and “B” a group having ingested wild-type rice as a control. The FIGS. 1, 2 and 3 on the abscissa in each graph indicate the next day after 4 times×2 administrations, the 7th day after 4 times×2 administrations and the next day after 4 times×3 administrations. Each coordinate indicates the serum anti-collagen antibody titer.

The present specification encompasses the content of the specification of Japanese Patent Application No. 2005-62996 on which the priority of the present application is based.

### BEST MODE FOR CARRYING OUT THE INVENTION

### 1. Glutelin Multigene Family

For the purpose of the present invention, “glutelin” is a kind of insoluble storage proteins of seed, not dissolving in water, salt solution and 70% alcohol. In a rice plant, the glutelin constitutes the bulk of the edible protein and is also called oryzenin. In addition, the glutelin is abundantly contained in wheat and barley and these glutelins are also referred to as glutenins. For the purpose of the present invention, “glutelin multigene family” shall include all of these glutelins without being limited to the origins and common names thereof.

Rice glutelin is a protein comprising two types of subunits having molecular weights of 37,000 and 22,000 to 23,000 (a basic subunit: glutelin A and an acidic subunit: glutelin B) and accounts for 70 to 80% of the storage protein. A glutelin gene is endosperm-specifically expressed; the tissue specificity is considerably strict and does not permit the expression thereof in other tissues such as the leaf and root. The rice glutelin gene group comprises about 10 genes per one haploid genome, and the genes are divided into two subfamilies: GluA encoding the basic subunit and GluB encoding the acidic subunit.

The base sequences of genes encoding the glutelin multigene family are already known and can be easily obtained through the public database GenBank or the like. For example, the cDNAs of the rice glutelins GluA and GluB have been deposited under the accession numbers XO5662, XO5661 and EO1546 (all of which are GluA) and X15833, AK107343, XI4568 (all of which are GluB), respectively. The cDNA sequence covering the whole region of ORF of GluA used in the present invention is shown in SEQ ID NO: 1.

### 2. Intended Peptide

For the purpose of the present invention, “intended peptide” is a peptide to be expressed and accumulated in a host plant; the type thereof is not limited. Use of the method of the present invention can efficiently and abundantly express and accumulate a low-molecular peptide which is usually difficult to stably express and accumulate in a plant, particularly in plant seeds. The low-molecular peptide used in the present invention is a peptide having an amino acid number of 3 to 40, preferably 3 to 30, more preferably 3 to 20.

A gene encoding an intended peptide (hereinafter referred to as “intended gene”) is ligated downstream of a gene encoding the glutelin multigene family member and expressed as a fusion protein with glutelin. In the vector of the present invention, the gene encoding an intended peptide is desirably subjected to the ligation of the repeated sequences of two or more, particularly 2 to 20 copies thereof. In this respect, “aspect capable of functioning” means that a transgene exerts a desired function in a host; for the purpose of the present invention, it means that an intended peptide is expressed as a fusion protein with glutelin in a plant.

Preferred examples of the intended peptide used in the present invention can include a T-cell epitope peptide of an antigenic protein in allergy or autoimmune disease (for example, type II collagen and 39 kDa cartilage glycoprotein in arthritis, pollen allergen Cry j1 in pollen disease, mite allergen Del I in asthma, or pancreatic β-cell antigen in diabetes), an antibacterial peptide (for example, defensin or lactoferricin), an antihypertensive peptide (ACE-inhibiting peptide), and an opioid peptide.

### 3. Construction of Vector

The vector of the present invention comprises a gene encoding the glutelin multigene family member and two or more copies of a gene encoding an intended peptide ligated to the gene encoding the member of the glutelin multigene family, under the control of a promoter.

The “vector” used in the present invention is not particularly limited provided that it can be replicated in a host; plasmid DNA, phage DNA, or the like may be employed. Examples of the plasmid DNA include a plasmid for an Escherichia coli host such as pBR322, pBR325, pUC118 and pUC119; a plasmid for Bacillus subtilis such as pUB110 and pTP5; a plasmid for a yeast host such as YEp13, YEp24 and YCp50; and a plasmid for a plant cell host such as pBI221 and pBI121. Examples of the phage DNA include λ phage. The vector may be a binary-type vector, which is, as described later, suitable to select a host free of selection marker from transformed plants.

The “promoter” used in the present invention is not particularly limited provided that it functions in host plant cells and allows the effective expression of an introduced trait of interest; examples thereof include cauliflower mosaic virus ³⁵S RNA promoter, rd29A gene promoter, rbcS promoter, glutelin A promoter, and glutelin B promoter. Among others, preferred are glutelin promoters having strong promoter activities (for example, GluB-1 promoter (GenBank Accession No. AY427569), GluB-2 promoter (GenBank Accession No. AY427570), GluB-4 promoter (GenBank Accession No. AY427571), and GluPF2 promoter (SEQ ID NO: 7)).

In order to appropriately express an intended peptide, cis elements such as an enhancer, a splicing signal, a poly(A) addition signal, a selection marker, a ribosome binding sequence (SD sequence), and the like may be, if desired, ligated to the vector in addition to the above-described promoter.

Examples of “terminator sequence” include terminators derived from cauliflower mosaic virus and nopaline synthase gene; however, the sequence is not limited thereto if it functions in a plant body.

As “selection marker”, a drug-resistance gene may be used, for example; a hygromycin-resistance gene, a bialaphos-resistance gene, or the like may be used when the plant is a rice plant. The selection marker is always considered problematic in terms of the safety of a recombinant plant. However, the marker is a gene necessary only for the efficient selection of transformed cells; after this, it makes no sense to leave the gene in the plant cells. Accordingly, the selection marker is desirably separated and removed in the selfed progeny of a plant transformed using a co-transformation vector, according to a method as already reported by the present inventors (see JP Patent Publication (Kokai) No. 2003-000082A).

As already reported, the co-transformation vector used may be a binary-type hybrid vector having two T-DNA regions. The vector is constructed of an intermediate vector containing an intended gene in one T-DNA region (a first T-DNA region) and an acceptor vector containing a selection marker in the other T-DNA region (a second T-DNA region). In the case of the present invention, it follows that the first T-DNA region contains two or more copies of a gene encoding an intended peptide composed of 3 to 40 amino acid residues ligated to a gene encoding a member of the glutelin multigene family.

In constructing the co-transformation vector, it is preferable to use a super binary-type vector (Hiei, Y. et al., 1994, Plant J., 6: 271-282) where the efficiency of introducing an intended gene is increased by placing, in a plasmid having T-DNA, a portion of the Vir region of a pathogenic bacterial strain having strong infectivity of Agrobacterium tumefaciens strains. Examples of the super binary-type vector can include vectors of pSB series (Japan Tobacco, Inc.: WO 95/16031 and Komari, T. et al., 1996, Plant J., 10: 165-174).

### 4. Transformation of Plant

The “plant” used in the present invention is not particularly limited; however, preferred is a rice plant, a wheat plant, a barley plant, a corn plant, a potato plant, a soybean plant, a rapeseed plant, a tomato plant, a banana plant, or the like in that the seeds thereof have high general versatility as a food. Particularly, the vector of the present invention can also be used in the useful rice variety “Koshihikari” which is difficult to transform and has a high commercial value.

The vector is introduced into a host plant by an ordinary method including a direct introduction method using electroporation or the like or an indirect introduction method through a bacterium of the genus Agrobacterium; preferred is the latter indirect introduction method through a bacterium of the genus Agrobacterium.

The form of the plant infected with a bacterium of the genus Agrobacterium is not particularly limited and can be properly selected from a callus, a leaf, a hypocotyl, a root, a seed, suspension cultured cells, protoplasts and the like depending on the system for redifferentiation of the plant. When the plant is a rice plant, calli derived from the scutellum of the rice plant are usually used; however, calli within about 3 weeks after induction from fully ripe seeds are preferably employed to obtain a stable and high gene introduction efficiency.

Particularly, calli from the rice variety “Koshihikari” are cultured under the following conditions. Specifically, as a callus induction medium, there is used a medium in which suitable amino acids are added to N6 basal medium whose nitrogen concentration is reduced (for example, KSP medium (Tsugawa et al., 1993, Ikushugaku Zasshi, 43 (suppl 2): 121)). In addition, 2 mg/L of 2,4-dichloroacetic acid (2,4-D) is used as a plant hormone; 30 g/L of maltose as a sugar; and 0.8% agarose as a coagulant. Surface-sterilized brown rice of variety “Koshihikari” is planted in the callus induction medium. Here, the endosperm portion is completely embedded in the medium with only the embryo portion exposed. A Petri dish is used as a container; the lid is covered with a vinyl tape having weak adhesion or the like to gradually promote the drying. The culture environment is a bright room at 28 to 30° C. This culture can induce fine granular calli suitable for gene introduction within 3 weeks from fully ripe seeds of rice variety “Koshihikari”.

The infection of the above forms of a plant with a bacterium of the genus Agrobacterium, the cocultivation, the bacteria elimination from the plant, the selection and growth of a transgenic plant, and the plant redifferentiation from the selected plant can be performed according to techniques known to those skilled in the art. However, when the plant is the rice variety “Koshihikari”, a method can be suitably used which has previously been developed by the present inventors (Hashizume et al., 1999, Plant Biotechnology, 16: 397-401).

### 5. Selection of Recombinant Plant Line Free of Selection Marker

As the above result, in a redifferentiated first generation (T0), a transgenic plant is produced which has intended gene (two or more copies of a gene encoding an intended peptide composed of 3 to 40 amino acid residues ligated to a gene encoding a member of the glutelin multigene family) and a selection marker. The resultant plant of the redifferentiated first generation (T0) is subsequently conditioned and cultivated to provide selfed progenies (T1 and T2) according to techniques known to those skilled in the art. Then, the DNAs of the differentiated first generation (T0), first selfed generation (T1) and second selfed generation (T2) are analyzed to finally select a recombinant plant line which is free of selection marker and has the intended gene in the form of a dominant homozygote.

### 6. Expression and Accumulation of Fusion Protein of Glutelin and Intended Peptide

The recombinant plant line selected in the preceding paragraph can be tested for the expression and accumulation of a fusion protein of glutelin and the intended peptide using an antibody specific to the intended peptide.

The method for detecting the protein using the antibody is not particularly limited; however, it is preferably any one selected from a Western blot method, a dot blot method, a slot blot method, an ELISA method and an RIA method.

The antibody used may be prepared according to a known method or may be a commercial antibody. The antibody can be obtained using conventional methods by immunizing an animal with an intended peptide forming an antigen or any polypeptide selected from the amino acid sequence thereof and collecting and purifying an antibody produced in the animal body. According to well known methods (e.g., Köhler and Milstein, Nature 256, 495-497, 1975, Kennet, R. ed., Monoclonal Antibody p. 365-367, 1980, Prenum Press, N.Y.), antibody-forming cells producing an antibody to the intended peptide can also be fused with myeloma cells to establish hybridoma cells, from which a monoclonal antibody is then obtained.

Examples of the antigen for preparing the antibody can include the intended peptide, a polypeptide composed of a continuous partial sequence of at least 6 amino acid residues thereof, or a derivative in which any amino acid sequence or carrier (e.g., keyhole limpet haemocyanin capable of addition to the N-terminal) is added thereto.

The antigen polypeptide can be obtained by allowing genetic engineered host cells to produce the intended peptide. Specifically, a vector capable of expressing the intended peptide may be prepared and introduced into host cells to express the gene. The resultant antibody is used for detection by directly labeling the antibody or by using the antibody as a primary antibody in combination with a labeled secondary antibody specifically recognizing the primary antibody (i.e., recognizing an antibody derived from an animal in which the antibody has been prepared).

Preferred examples of the type of the label include, but not limited to, an enzyme (alkaline phosphatase or horseradish peroxidase) or biotin (wherein an enzyme-labeled streptavidin is additionally bound to the biotin in the secondary antibody). Various prelabeled antibodies (or streptavidins) are commercially available as the labeled secondary antibody (or the labeled streptavidin). In the case of RIA, an antibody labeled with a radioactive isotope such as ¹²⁵I is used; the measurement is carried out employing a liquid scintillation counter or the like. The enzymatic activities of these enzyme labels are each detected to determine the expression level of the antigen. When the antibody is labeled with alkaline phosphatase or horseradish peroxidase, substrates are commercially available which develop color or emit light by the catalysis of these enzymes.

Use of the substrate developing color makes visual detection possible by employing the Western blot method or the dot/slot blot method. In the ELISA method, it is preferable to measure and quantitate the absorbance in each well (the measuring wave length varies depending on the substrate) using a commercially available microplate reader. A dilution series of the antigen used for producing the antibody can also be prepared, used as a standard antigen sample and subjected to a detection operation simultaneously with a different sample to quantitate the antigen concentration in the different sample by making a standard curve on which the measured values are plotted against the standard antigen concentrations.

Use of the substrate emitting light can allow the detection by photography with an instant camera or autoradiography using an X-ray film or imaging plate in the Western blot method or the dot/slot blot method. The quantitative analysis is also possible using densitometry or Molecular Imager Fx System (from Bio-Rad Laboratories, Inc.). In addition, when the light-emitting substrate is used in the ELISA method, the enzyme activity is measured employing an emission micro plate reader.

By the foregoing methods, the present inventors have confirmed that a fusion protein of glutelin and an intended peptide is highly expressed and accumulated in seeds of the recombinant plant of the present invention. The present invention provides not only the recombinant plant but also cells, tissues or organs of the plant or cultures thereof.

The above cells, tissues and organs include all of the cells, tissues and organs in all differentiation processes in a plant. Specifically, the cells may be single cells or an aggregate (a mass of cells) and also include protoplasts and spheroplasts. The tissues may also be single tissues or an aggregate and include all tissues such as epidermal tissues, parenchymas, phloem tissues (e.g., sieve tubes, phloem fiber) and xylem tissues (e.g., vessels, tracheids, xylem fiber). The organs also include all organs such as the stem, tuber, leaf, root, tuberous root, scion, bud, flower, petal, pistil, stamen, anther, pollen, ovary, fruit, pod, capsule, seed, fiber and ovule. Among others, the seed in which a fusion protein of an intended peptide and glutelin accumulates can have a high utility value as a functional food, as described later.

A plant of the present invention can also be regenerated, according to an ordinary method, from a culture of the above cells, tissue or organ (e.g., an embryo culture, an ovule culture, an ovary culture, an anther culture, a shoot apex culture, a pollen culture).

### 7. Multiple Introduction of Intended Gene into Single Gene Locus

The vector of the present invention comprising two or more ligated copies of an intended gene provides a high expression line with higher probability than a vector comprising a single copy of the gene. This is not ascribed merely to that the vector contains a number of copies of the intended gene but attributed to that the vector multiply infects a single gene locus to multiply introduce two or more ligated copies of the intended gene into the single gene locus. Although the detailed mechanism is uncertain, the repeated sequences in the vector are suggested to make some contribution because the probability of multiple infection becomes higher and the prevalence of a high expression line increases as the number of copies (repeat number) of the intended gene contained in the vector is increased.

The location of the introduced genes on the host genome is generally random and difficult to artificially control; thus, it has not been easy to obtain a high expression multicopy line by a conventional method. Use of the vector of the present invention enables multiple copies of a gene to be collectively introduced into a narrow region (a single gene locus) of the host genome, which permits the easy production of a multicopy line which is genetically stable and shows high expression.

### 8. Use of Recombinant Plant of the Present Invention

According to the present invention, a low-molecular peptide can be stably and highly expressed and accumulated which is usually difficult to stably express and accumulate in a plant, particularly in plant seeds. Thus, if a peptide having a physiological function useful in maintaining and improving health is selected as an intended peptide to develop a plant seed in which the peptide is highly expressed and accumulated, the seed can be used as a medicine or functional food for assisting the prevention or treatment of a food-born disease such as cardiac disease, hypertension and allergy. Examples of the peptide can include a I-cell epitope peptide of an allergen or a causative antigen in autoimmune disease (for example, type II collagen epitope), an antibacterial peptide (for example, defensin or lactoferricin), an ACE-inhibiting peptide (some peptides have already been registered as special health food), and an opioid peptide (analgetic peptide).

The present invention describes an example of preparing a rice plant expressing a tolerogenic (epitope) peptide of human type II collagen as an example of the medicine or functional food.

### EXAMPLES

The present invention is described below in detail by way of Examples.

### Example 1

### Construction of Fusion Gene for Expressing Type II Collagen Peptide

### 1. Construction of Vector for Expressing Type II Collagen Peptide

The amino acid sequence of a T-cell recognition epitope region peptide of human type II collagen (HuCII) (SEQ ID NO: 3) was converted into a base sequence employing optimal codons for a rice plant (SEQ ID NO: 4). Primers described below were designed based on the resultant sequence so that a SalI site was added upstream of HuCII gene and a tyrosine (Tyr) sequence and a XhoI site are added downstream thereof. The primers were annealed using Klenow fragment to artificially synthesize HuCII gene. Sequencing demonstrated that the HuCII gene had a correct sequence. The both ligation potions of SalI site and the XhoI site have a common sticky-end sequence of TCGA and therefore can be ligated each other between SalI and XhoI sites. Clones in which HuCII genes were ligated to make palindromic linkages between the SalI sites and between the XhoI sites were cut apart by SalI-XhoI treatment to form monomeric HuCII genes; these clones were discarded because they could be determined as clones in which two or more HuCII genes were ligated together in the wrong direction. On the other hand, a clone not fragmented by the two restriction enzymes was selected as a clone in which the two or more HuCII genes were ligated together in the correct direction. The above operation was repeated to synthesize cDNAs in each of which 4, 8 or 16 copies of HuCII gene were ligated together. Sequencing demonstrated that these cDNAs were correct in the direction of ligation and the sequence. In this respect, the cloning of each cDNA was performed using pBluescript.

(SEQ ID No: 5)

| | |
|---|---|
| Forward primer: | 5′-ATgTCgACggCCCAAAgggCCAgACCggCAA |
| | gCCAggCATCgCCggCTTCA-3′ |

(SEQ ID No: 6)

| | |
|---|---|
| Reverse primer: | 5′-ATCTCgAgATACTTTgggCCCTgCTCgCCCT |
| | TgAAgCCggCgATgCCTggC-3′ |

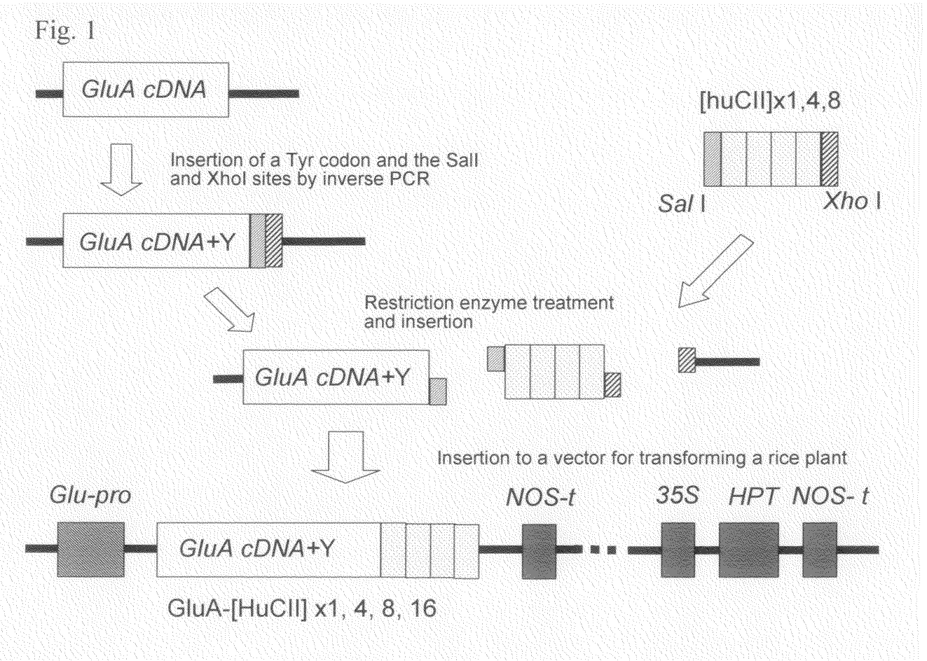

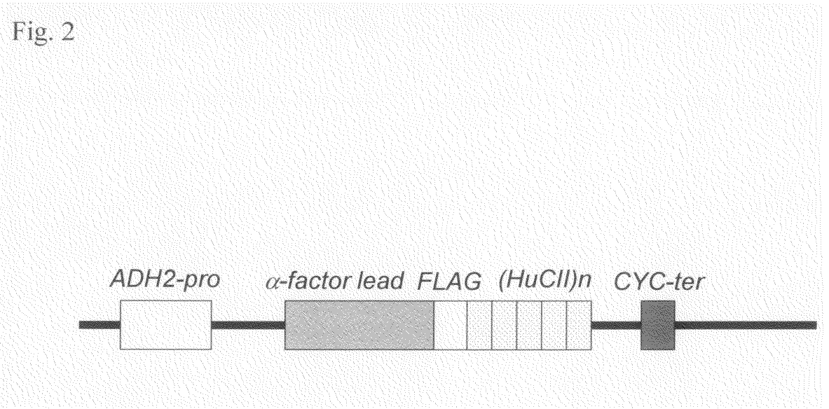

A SalI site-XhoI site-stop codon-SacI site was inserted downstream of glutelin A cDNA (GluA (SEQ ID NO: 1, a gift from Japan Tobacco, Inc.)) by inverse PCR using Pyrobest DNA polymerase (from Takara Bio Inc.). Sequencing demonstrated that the modification region was correctly inserted and that there was no change in the sequence of the glutelin A cDNA. A fusion gene was prepared in which 1, 4 or 8 copies of HuCII genes were ligated to the SalI site-XhoI site downstream of the glutelin A cDNA (the first half of FIG. 1). Insertion in the correct direction was confirmed by the separation of the glutelin A cDNA and the HuCII gene by SalI-XhoI treatment. In addition, the cDNA comprising 8 or 16 ligated copies of HuCII genes alone or the cDNA comprising 8 ligated copies of HuCII genes fused to the glutelin gene was inserted into YEpFLAG, a vector for secretion in yeast (from Sigma) (FIG. 2).

### 2. Preparation of Type II Collagen Peptide by Yeast and Production of Specific Antibody

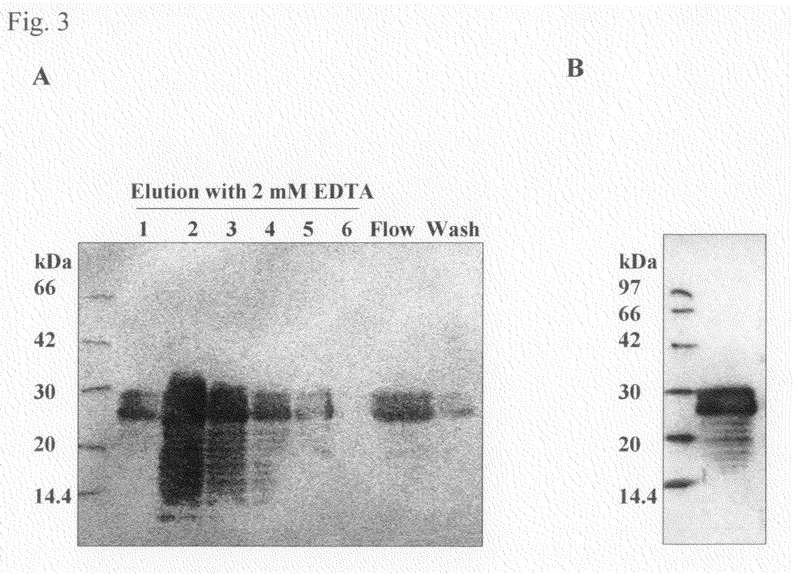

A yeast was transformed with the prepared vector; extracellularly secreted HuCII was immunochemically detected and identified using an anti-FLAG antibody. A high expression line was selected and subjected to large-scale culture; the culture solution was concentrated and purified by anti-FLAG antibody affinity chromatography (FIG. 3). The purified recombinant HuCII was used as an antigen to immunize mice to produce an antibody specific thereto.

About 0.5 mg of [HuCII]×8 was obtained by purification from 800 ml of the culture supernatant of the transformed yeast by anti-FLAG antibody affinity chromatography. The purified recombinant [HuCII]×8 was used as an antigen to immunize mice to provide an antibody specific thereto.

Western analysis was then performed with the antibody. According to the Laemmli method, a sample (equivalent to 20 μg) was placed on a 12.5% SDS polyacrylamide gel and subjected to electrophoresis at the constant current of 40 mA for 45 minutes. Then, protein was transferred to an electrophoresis transference membrane (Clear Blot Membrane P, from ATTO) by semidry blotting. [HuCII] was detected using the ECL Western blot detection system (from Amersham Bioscience). As a result, [HuCII]×8 in the culture supernatant and [HuCII]×8 and [HuCII]×16 in the yeast cells were detected at high sensitivity.

### 3. Construction of Vector for Co-Transformation

Three types of vectors for co-transformation, each containing [HuCII]×1, [HuCII]×4 or [HuCII]×8 were prepared according to the method described in JP Patent Publication (Kokai) No. 2003-000082A using the super binary vector pSB424 (a gift from Japan Tobacco, Inc.; WO95/16031 and Komari, T. et al., 1996, Plant J., 10: 165-174). The pSB424 is a hybrid vector provided by homologous recombination between the intermediate vector pSB24 and the acceptor vector pSB4 (both of which were obtained from Japan Tobacco, Inc. through contract distribution.).

The intermediate vector pSB24 was digested with HindIII-XbaI, and the CaMV35S promoter therebetween was replaced with the glutelin promoter GluPF2 (SEQ ID NO: 7) digested with the same restriction enzyme (the resultant vector was designated as pSB26). The pSB26 was digested with BamHI-SacI, and the GUS reporter gene therebetween was replaced with the glutelin A cDNA-[HuCII]×1, 4 or 8 fusion gene digested with the same restriction enzyme (pSB26Glu-Cn, n=1, 4 or 8) (FIG. 1).

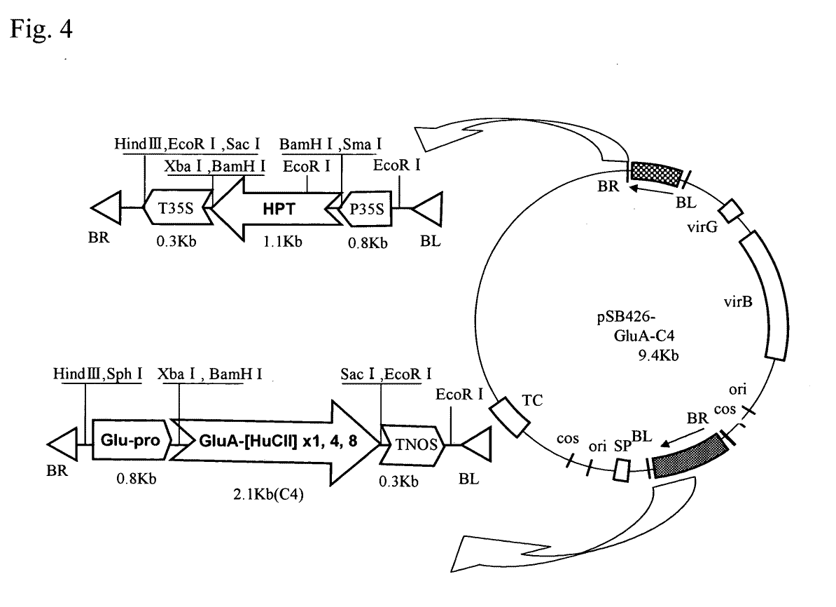

Subsequently, the three types of bacteria, the Agrobacterium LBA4404 containing the acceptor vector pSB4, the Escherichia coli LE392 containing the intermediate vector pSB26Glu-Cn, and the Escherichia coli HB101 containing the helper plasmid pRK2013, were mixed together on Nutrient Agar (from Difco) and subjected to cocultivation at 28° C. overnight. After the cultivation, an operation was repeated several times in which the mixed lines were thinly stripe-seeded on AB medium (Chilton, M.-D. et al., 1974, Proc. Natl. Acad. Sci., USA, 71: 3672-3676) containing 50 mg/L of spectinomycin and 50 mg/L of hygromycin to select a clone resistant to both of the antibiotics. The clone was an Agrobacterium containing the hybrid vector pSB426Glu-Cn combining pSB4-derived hygromycin resistance and pSB26Glu-Cn-derived spectinomycin resistance, and was designated as LBA4404/pSB426Glu-Cn (n=1, 4, or 8). The structure of the vector pSB426Glu-C4 is shown in FIG. 4.

### Example 2

### Obtaining Transformed Rice Plant

### 1. Transformation of Rice Plant with pSB426Glu-Cn

Fully ripe seeds of rice variety “Koshihikari” (Oryza sativa L. var Koshihikari) were surface-sterilized and then planted in a KA-1 medium (which was based on KSP medium and contained 2 mg/L of 2,4-D, 30 g/L of maltose, and 0.8% agarose), followed by sealing the Petri dish with vegetable binding tape (from Nitto Denko Corporation) before culture in a bright room at 28° C. After 3 weeks, many fine granular calli having high mitogenic activity were induced.

The calli were infected with LBA4404/pSB426Glu-Cn (n=1, 4, and 8) and subjected to drug selection and redifferentiation according to a method of Hashizume et al. (1999) to provide 100 rice transformants (T0). The specific operation is described below.

One spoon of cells of the Agrobacterium grown on AB medium (Chilton, M.-D. et al., 1974, Proc. Natl. Acad. Sci., USA, 71: 3672-3676) containing 50 mg/L of hygromycin was taken using a microspatula and sufficiently suspended in 20 mL of KA-1 liquid medium (which was based on KSP medium, in which the amount of 2,4-D was modified to 2 mg/L and sucrose was changed to 30 g/L of maltose; pH 5.8) containing 10 mg/L of acetosyringone to such a degree that bacterial blocks came loose with the suspension becoming uniform. The suspension was transferred to a 9-cm glass Petri dish. The induced calli were then placed in a stainless mesh (mesh size: 20 mesh) shaped into a basket, followed by soaking the mesh in the bacterial suspension for one minute and 30 seconds so that the whole calli were immersed. After removing the bacterial suspension, the calli were transferred onto sterilized filter paper to get rid of the excess water. Two superposed sheets of sterilized filter paper were placed on KA-1co medium (in which 10 g/L of glucose and 10 mg/L of acetosyringone were added to KA-1 medium and which contained 1.5% bactoagar; pH 5.2); the calli were placed thereon so that they did not overlap with each other. After subjecting the calli to co-cultivation in a dark room at 28° C. for 3 days, the excess bacterial cells were washed away from the calli with sterile water to such a degree that the solution was clear; then, the calli were rinsed with KA-1 liquid medium containing 250 mg/L of carbenicillin. The calli was drained with sterilized filter paper and placed on KA-1 se medium (in which 250 mg/L of carbenicillin and 50 mg/L of hygromycin were added to KA-1 medium and which contained 0.8% agarose; pH 5.8). The calli were cultured in a bright room with a 14-hour day length at 28 to 30° C. for 3 weeks, followed by transplanting all thereof into fresh KA-1se medium. After 2 to 3 weeks, the calli surviving and growing under selection with hygromycin were placed on KA-2 medium (in which 30 g/L of sorbitol, 2 g/L of casamino acid, 125 mg/L of carbenicillin, and 50 mg/L of hygromycin were added to KA-1 medium in which the plant hormone was changed to 0.4 mg/L of 2,4-D, 0.5 mg/L of abscisic acid (ABA), and 0.1 mg/L of kinetin, and which contained 0.8% agarose; pH 5.8) for one week. Then, the calli were transplanted into KA-3 medium (which was KA-2 medium in which the plant hormone was changed to 0.5 mg/L of 6-benzylaminopurine (BAP) and 0.2 mg/L of indoleacetic acid (IAA) and the hygromycin concentration was modified to 25 mg/L and which contained 0.8% agarose; pH 5.8) and redifferentiated into plants in 3 to 4 weeks.

The T0 plants were identified for the presence of the transgene by PCR. Then, DNA was extracted from the leaves thereof and subjected to PCR analysis using primers for HuCII detection as described below. The transgene deletion lines were discarded; after flowering, the ripening lines (those from each of which 50 or more T1 seeds could have been obtained) were further selected.

| | |
|---|---|
| Forward primer: | |
| 5′-CTCAGAGGCTCAAGCATAATAGAGG-3′ | (SEQ ID No: 8) |
| Reverse primer: | |
| 5′-GAGCTCCTACTCGAGATACTTTGGG-3′ | (SEQ ID No: 9) |

As a result, as first selfed generation (T1), there were obtained 73 plants having GluA-[HuCII]×1, 33 having [HuCII]×4, and 63 having [HuCII]×8.

### 2. Detection of GluA-[HuCII] Fusion Protein

After efflorescence, salt-soluble and -insoluble proteins were extracted from the seeds of first selfed generation (T1); the HuCII continued therein was analyzed by a Western method using the antibody to recombinant HuCII prepared previously.

To each of sample rices was first added 200 μl of a PBS solution (pH 7.5), which was subjected to a grinder (MM-300 from Quiagen, frequency: 30 rpm, 1 minute×2 times) and shaken at 4° C. and 100 rpm for 5 minutes, followed by subjecting to a centrifuge (15,000 rpm, 3 minutes) before discarding the supernatant. To the resultant precipitate was added 400 μL 2× sample buffer, which was treated at 100° C. for 3 minutes to extract the proteins. The extracted proteins (4 μL each) were subjected to SDS-PAGE (phoresis bath AE-6500 from ATTO; 15% gel; 40 mA, phoresis for 35 minutes) for separation, transferred to an electrophoresis transference membrane (Clear Blot Membrane P, from ATTO), immunostained with an HuCII-specific antibody and an enzyme-labeled secondary antibody, and then analyzed using the ECL Western blot detection system (from Amersham Bioscience).

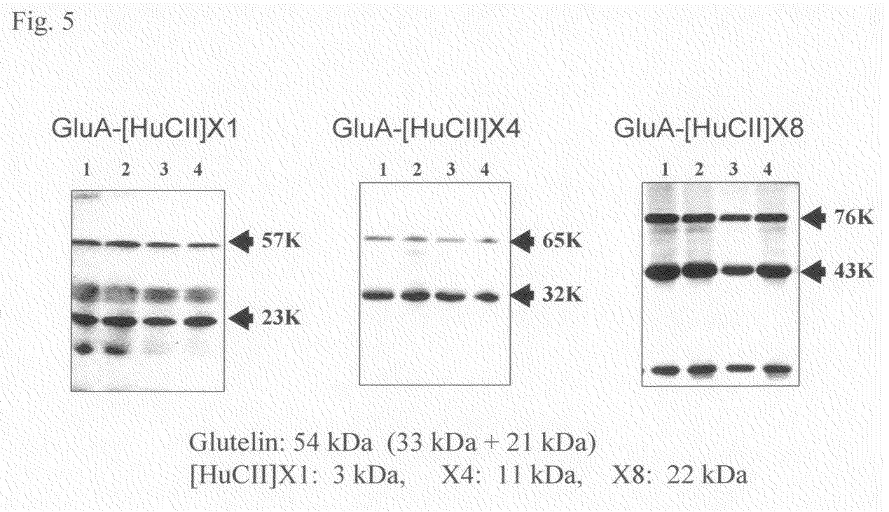

As a result, GluA-[HuCII] was detected only in the insoluble protein fraction of the endosperm in T1 seeds of each line. Bands deduced to be an about 60 kDa precursor and an about 35 kDa mature form were detected for GluA-[HuCII]×4 and ×8, while a band estimated to be a degradation fragment of 20 kDa or less was detected for GluA-[HuCII]×1 (FIG. 5).

### Example 3

### Removal of Selection Marker in Transformed Plant

### 1. Seed Protein Analysis in First Selfed Generation (T1) of Redifferentiated Plants

The proteins of T1 seeds were analyzed on a per seed basis by a Western analysis method using an antibody specific to a human type II collagen peptide to select a redifferentiated first generation (T0) line in which a seed expressing an [HuCII]-glutelin fusion protein was found with a high frequency. Specifically, 42 plants having GluA-[HuCII]×1, 14 having GluA-[HuCII]×4, and 35 having GluA-[HuCII]×8 were selected in a primary screening. In addition, 15 plants having GluA-[HuCII]×1, 12 having GluA-[HuCII]×4, and 21 having GluA-[HuCII]×8 were selected in a secondary screening.

### 2. Half-Seed Protein Analysis of T1 Seed and Genetic Analysis of Half-Seed-Derived Second Selfed Generation (T2)

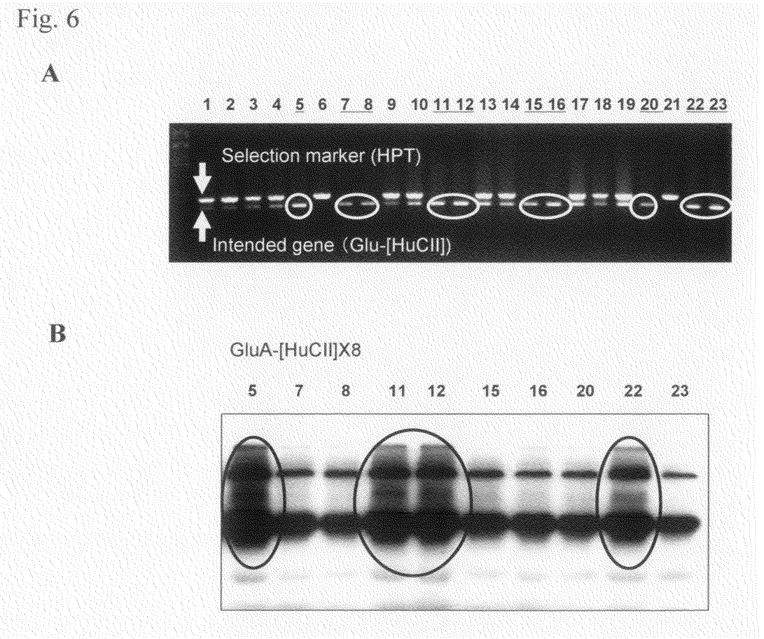

When Western analysis is performed using the whole T1 seed as a material therefor, a plant (T1) cannot be grown from the seed from which the results of the analysis have been obtained. Accordingly, the seed was divided into two halves (half-seeds), which were then used for Western analysis and the genetic analysis of a seedling therefrom to narrow down T0 lines to those in which the two genes were probably separated and monofactorially inherited (FIG. 6). Use of this half-seed analysis method can not only reserve a next-generation plant, but also employ PCR analysis and Western analysis in combination to make more efficient the selection.

Specifically, T1 half-seeds (50 to 80 seeds per line) containing the embryo were each germinated. DNA was then extracted from the seedling of the T1 plant and analyzed by PCR for the presence of HuCII and HPT genes, thereby discriminating seeds having HuCII gene but no HPT gene. In addition, the half-seeds free of the embryo were used for Western analysis and identified for the expression of HuCII to provide promising T0 lines having any of three types of genes ([HuCII]×1, ×4 and ×8): 4 lines having GluA-[HuCII]×1, 3 having GluA-[HuCII]×4, and 3 having GluA-[HuCII]×8. The estimated results for the hereditary of T0 lines by T1 seedling analysis are collectively shown in Table 1.

TABLE 1
Estimation of hereditary of T0 lines by analysis of T1 seedling

| Sample | Number | No. | Number of analyses | g/h | g/— | —/h | —/— | Number of loci | T1 cultivation |
|---|---|---|---|---|---|---|---|---|---|
| GluA-C8 | T(2) | 1 | 48 | 30 | 0 | 0 | 18 | g-h(1) | ◯ |
| GluA-C8 | | 4 | 48 | 39 | 0 | 0 | 9 | g-h(1) | X |
| GluA-C8 | | 7 | 16 | 11 | 0 | 0 | 5 | g-h(1) | X |
| GluA-C8 | T(5) | 6 | 48 | 32 | 0 | 0 | 16 | g-h(1) | X |
| GluA-C8 | | 7 | 16 | 13 | 0 | 0 | 3 | g-h(1) | X |
| GluA-C8 | | 13 | 48 | 36 | 0 | 0 | 12 | g-h(1) | ◯ |
| GluA-C8 | | 16 | 48 | 37 | 0 | 0 | 11 | g-h(1) | X |
| GluA-C8 | | 19 | 16 | 13 | 0 | 0 | 3 | g-h(1) | X |
| GluA-C8 | | 21 | 25 | 18 | 0 | 5 | 2 | Non-specific | X |
| GluA-C8 | | 22 | 22 | 16 | 0 | 0 | 6 | g-h(1) | X |
| GluA-C8 | | 26 | 16 | 10 | 0 | 0 | 6 | g-h(1) | X |
| GluA-C8 | | 27 | 81 | 54 | 21 | 4 | 2 | g(2), h(1) | ⊚ |
| GluA-C8 | T(8) | 5 | 48 | 35 | 0 | 0 | 13 | g-h(1) | X |
| GluA-C8 | | 8 | 82 | 65 | 15 | 0 | 2 | g(1), g-h(1) | ⊚ |
| GluA-C8 | | 9 | 48 | 40 | 0 | 0 | 8 | g-h(1) | ◯ |
| GluA-C8 | | 10 | 48 | 38 | 0 | 0 | 10 | g-h(1) | ◯ |
| GluA-C8 | | 22 | 83 | 63 | 16 | 0 | 4 | g(1), g-h(1) | ⊚ |
| GluA-C8 | | 24 | 16 | 16 | 0 | 0 | 0 | Multifactor | X |
| GluA-C8 | | 27 | 16 | 16 | 0 | 0 | 0 | Multifactor | X |
| GluA-C8 | | 44 | 22 | 20 | 1 | 1 | 0 | Multifactor | X |
| T1(GluA-C8 | | | 795 | | | | | | |
| Subtotal | | | | | | | | | |
| GluA-C4 | T(1) | 2 | 80 | 62 | 13 | 0 | 5 | g(1), g-h(1) | ⊚ |
| GluA-C4 | | 4 | 56 | 46 | 0 | 0 | 10 | g-h(1) | ◯ |
| GluA-C4 | | 8 | 24 | 24 | 0 | 0 | 0 | Multifactor | X |
| GluA-C4 | | 13 | 25 | 25 | 0 | 0 | 0 | Multifactor | X |
| GluA-C4 | | 14 | 60 | 46 | 10 | 0 | 4 | g(1), g-h(1) | ⊚ |
| GluA-C4 | | 19 | 23 | 23 | 0 | 0 | 0 | Multifactor | X |
| GluA-C4 | | 25 | 74 | 72 | 0 | 2 | 0 | Multifactor | X |
| GluA-C4 | | 26 | 73 | 64 | 6 | 1 | 2 | Multifactor | X |
| GluA-C4 | | 33 | 24 | 24 | 0 | 0 | 0 | Multifactor | X |
| GluA-C4 | | 40 | 24 | 23 | 0 | 1 | 0 | Multifactor | X |
| GluA-C4 | | 44 | 80 | 60 | 17 | 0 | 3 | g(1), g-h(1) | ⊚ |
| T1(GluA-C4) | | | 543 | | | | | | |
| Subtotal | | | | | | | | | |
| GluA-C1 | T(3) | 16 | 66 | 61 | 5 | 0 | 0 | Multifactor | X |
| GluA-C1 | | 22 | 72 | 49 | 10 | 10 | 3 | g(1), h(1) | ⊚ |
| GluA-C1 | T(6) | 21 | 83 | 63 | 15 | 0 | 5 | g(1), g-h(1) | ⊚ |
| GluA-C1 | | 37 | 84 | 62 | 13 | 0 | 9 | g(1), g-h(1) | ⊚ |
| GluA-C1 | | 44 | 82 | 71 | 10 | 0 | 1 | g(2), g-h(1) | ⊚ |
| GluA-C1 | T(9) | 1 | 21 | 21 | 0 | 0 | 0 | Multifactor | X |
| GluA-C1 | | 7 | 53 | 49 | 0 | 0 | 4 | g-h(2) | X |

| | |
|---|---|
| T1(GluA-C1) | 461 |
| Subtotal | |
| Analysis T1 total | 1799 |

In the table, GluA-C1, GluA-C4, and GluA-C8 indicate GluA-[HuCII]×1, GluA-[HuCII]×4, and GluA-[HuCII]×8, respectively.

For T1 seeds of three promising T0 lines, a HuCII gene-positive line was subjected to half-seed Western analysis. As a result, although HuCII was expressed in all of the half-seeds, the expression level varied by half-seed. From this, the presence of homozygous and heterozygous T1 seeds was deduced.

### 3. Comparative Analysis of Prevalence of Marker Gene Separation-type Line

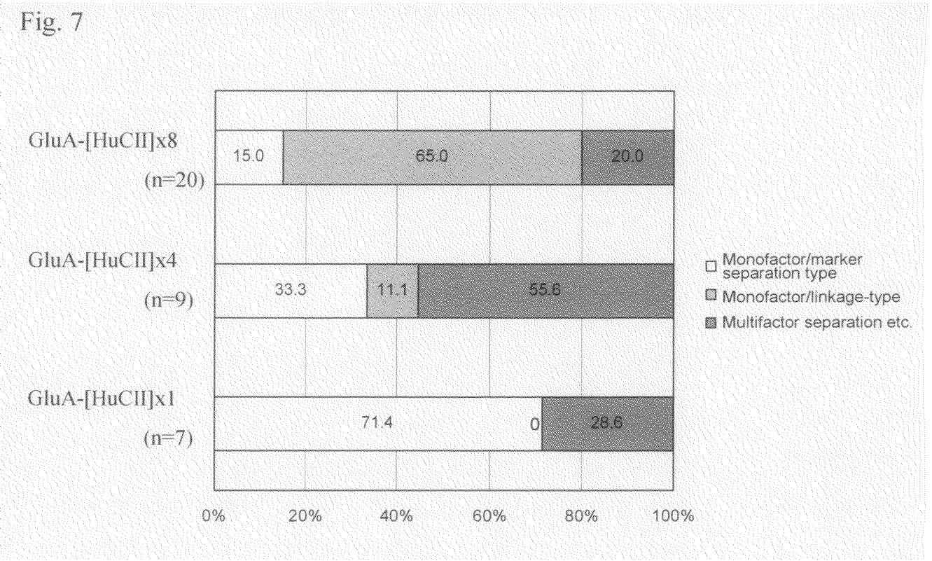

In transformed rice plants (redifferentiated first generation, T0) into each of which any of three types of genes ([HuCII]×1, ×4 and ×8) was introduced, it was estimated, from inheritance to T1 generation, whether HuCII and drug resistance genes are linked or separated, and the prevalences of the linkage- and separation-type lines were compared. The proportion of lines in which the two genes were estimated to be integrated in a separated form was most high for [HuCII]×1 (C1); the proportion of the integration in a separated form was decreased as the number of the peptide-encoding gene ligated together was increased to 4 and 8 (FIG. 7). In other words, many of the [HuCII]×1 (C1)-introduced lines were, in theory, “separation-type” lines into each of which HuCII and drug resistance genes were introduced at different gene loci, while many of the [HuCII]×8 (C8)-introduced lines were, conversely, “linkage-type” lines in which the [HuCII]×8 (C8) was inherited in linkage with the drug resistance gene, providing contrasting results.

Further, in the HuCII×4- and HuCII×8-introduced lines, comparison of the expression level of the HuCII peptide showed that the level was higher in linkage-type lines thereof than in separation-type lines thereof. From this, it was probable that HuCII×4 or HuCII×8 was multiply introduced into the linkage-type line. These results suggest that for a common gene, with a high frequency, the introduction thereof is conducted in a separated form, i.e., a single gene is introduced into one gene locus, while for a gene having repeated sequences (HuCII×4 or HuCII×8), with a high frequency, a plurality of molecules of the gene are multiply introduced at once into one locus.

For [HuCII]×8-introduced lines, a plurality of HuCII gene-homozygous T1 lines were obtained for which the HuCII gene was detected in all of the T2 plants thereof analyzed. In addition, comparison of the results of half-seed protein analysis of T1 seeds and the results of genetic analysis of T2 population demonstrated that the homozygosity or heterozygosity of the HuCII gene for the T1 seed could be deduced with a relatively high degree of accuracy from the expression level of the protein in the seed (Table 2).

TABLE 2
Selection of T1-fixed line by analysis of T2 seedling

| | | | | | | | Check |
|---|---|---|---|---|---|---|---|
| | | T1 half- | Number | | | | against |
| T1 line | | seed | of T2 | | | Deduced | half-seed |
| number | | western | analyses | g/— | —/— | fixed line | analysis |
| 808- | 1 | n.t. | 13 | 9 | 4 | Heterozygous | — |
| | 13 | n.t. | 15 | 15 | 0 | Homozygous | — |
| | 14 | n.t. | 15 | 11 | 4 | Heterozygous | — |
| | 18 | n.t. | 14 | 9 | 5 | Heterozygous | — |
| | 25 | n.t. | 15 | 15 | 0 | Homozygous | — |
| | 26 | High | 14 | 14 | 0 | Homozygous | ◯ |
| | 36 | High | 14 | 14 | 0 | Homozygous | ◯ |
| | 51 | High | 14 | 7 | 7 | Heterozygous | X |
| | 52 | Low | 12 | 8 | 4 | Heterozygous | ◯ |
| | 55 | High | 12 | 12 | 0 | Homozygous | ◯ |
| | 63 | Low | 12 | 11 | 1 | Heterozygous | ◯ |
| | 65 | High | 14 | 14 | 0 | Homozygous | ◯ |
| | 74 | Low | 13 | 9 | 4 | Heterozygous | ◯ |
| | 77 | High | 15 | 15 | 0 | Homozygous | ◯ |

Based on the above results, lines could be selected which contain the HuCII gene in the form of a homozygote and are free of the drug resistance gene (selection marker).

### Example 4

### Accumulation of Glutelin-HuCII Fusion Protein in Seed

### 1. Accumulation of Expressed HuCII Fusion Protein in Endosperm Protein Body

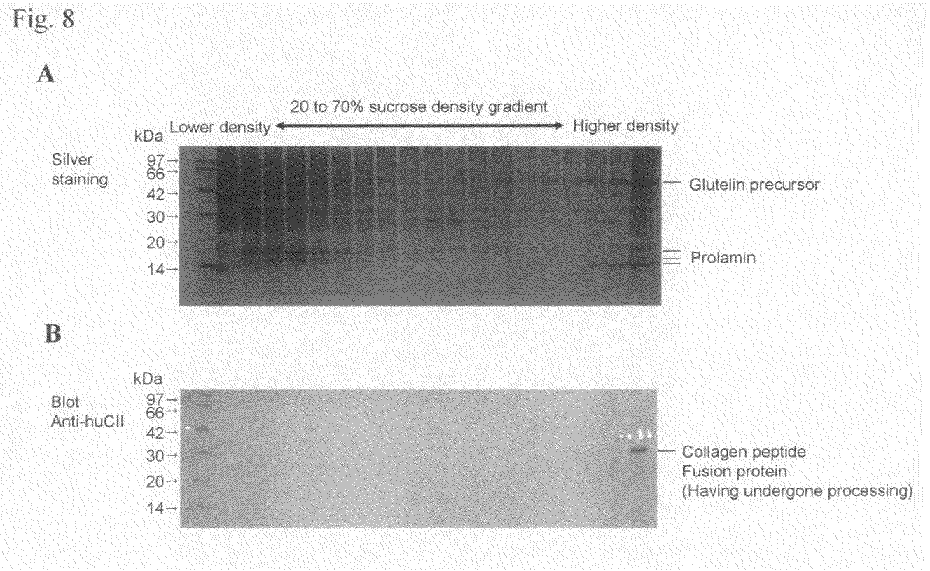

To establish whether the expressed glutelin-HuCII fusion protein accumulated in the protein body of the endosperm or not, the endosperm of ripening seeds was fractionated by sucrose density gradient ultracentrifugation, followed by determining the presence of the glutelin-HuCII fusion protein in each fraction by Western analysis. As a result, it was demonstrated that the desired glutelin-HuCII fusion protein was detected only in the same high density fraction as that for endogenous glutelin and that the fusion protein also accumulated in the protein body (FIG. 8).

### 2. Estimation of Content of Expressed HuCII Fusion Protein in Seed

The protein of each seed ripened by a GluA-[HucII]×8 homozygous plant was quantitatively extracted and subjected to semiquantitation using SDS-PAGE and a Western blot method. The [HuCII]×8 expressed in Escherichia coli was purified, and a solution thereof whose protein concentration had been determined by BCA assay was used as a standard product. The protein extracted from one seed and the standard product were electrophoresed in the same gel, transferred to the same PVDF membrane, and then detected by an ECL method using an [HuCII]×8-specific antibody. The signal intensity of each band was quantified using Densitograph (from ATTO), followed by calculating the concentration of [HuCII]×8 in the extract therefrom on the basis of known concentrations of the standard product. As a result, the content of [HuCII]×8 per seed was estimated to be at a level of 0.5 to 1.0 microgram.

The amount of HuCII ingestible at a single meal (assuming a bowlful of boiled rice=4000 seeds) is about 2 to 4 mg when calculated from the expression level per seed. According to past clinical reports, the oral ingestion of type II collagen at a level of micrograms per day led to a tendency of inducing immunotolerance in rheumatic patients (Choy E H, et al., Arthritis Rheum, 2001 September; 44(9): 1993-7, Barnett M L., Arthritis Rheum. 1998 February; 41(2): 290-7). Thus, it was corroborated that the rice plant prepared in the above Example accumulated, in the endosperm, the glutelin-HuCII fusion protein enough to exert an anti-rheumatic effect (immunotolerance) in usual dietary intake.

### Example 5

### Southern Blot Analysis of Marker Gene Separation-Type High Expression Line

Of the T1 fixed lines free of a marker obtained in Example 3, the lines having a particularly high expression level of peptide ([HuCII]×1 (C1)-introduced line: No. 322-1 and [HuCII]×4 (C4)-introduced lines: Nos. 527-41, 808-36 and 102-28) were analyzed for the proteins expressed in the seeds thereof by a Western method according to the procedure in Example 2. In this respect, the already established high expression T1 and non-expression T1 lines were used as a positive control (PC) and a negative control (NC), respectively.

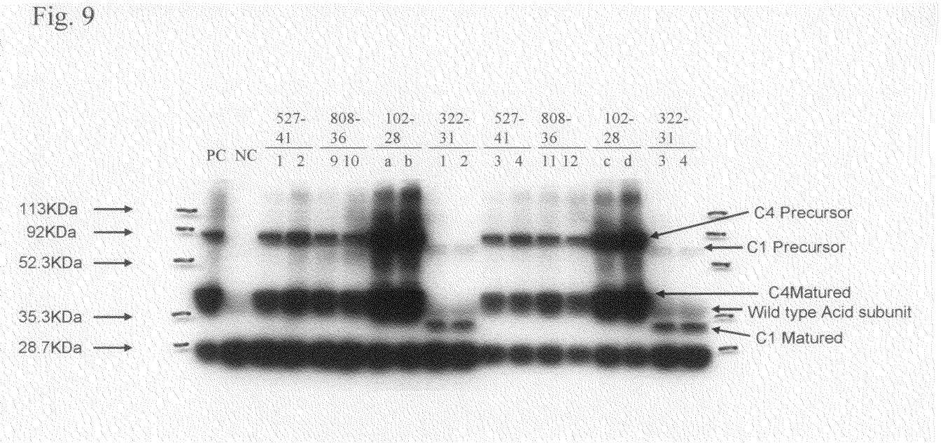

The results are shown in FIG. 9. In the figure, C1 Precursor indicates a fusion protein of glutelin A and [HuCII]×1 having not undergone processing (limited degradation) (precursor); C4 Precursor, a fusion protein of glutelin A and [HuCII]×4 having not undergone processing (limited degradation) (precursor); C1 Matured, a fusion protein of glutelin A and [HuCII]×1 having undergone processing (limited degradation) (mature-form); C4 Matured, a fusion protein of glutelin A and [HuCII]×4 having undergone processing (limited degradation) (mature-form); and Wild type Acid subunit, an acidic subunit of endogenous glutelin (mature-form glutelin having undergone limited degradation). As a result, it was demonstrated that the drug resistance gene was segregated in both of the [HuCII]×4-introduced 529-41 and 808-36 lines, which expressed and accumulated a markedly larger amount of the HuCII peptide than the [HuCII]×1-introduced 322-31 line. The difference in the expression level was 4-fold or more, and was probably not due simply to the 4-linkage but due to the multiple introduction of the intended gene into one gene locus. In addition, although the 102-28 line was of maker-linkage type, the line showed much higher levels of expression and accumulation than the other two lines (529-41 and 808-36) despite that it was a line into which the same [HuCII]×4 was introduced.

Southern analysis was further performed for the No. 102-28 line having an anomalously high expression level of the HuCII peptide of the above T1 fixed lines and its progenies T2 and T3. The Southern analysis was carried out by the following procedure according to the DIG application manual from Roche. Genome DNA was first extracted from 200 to 300 mg of rice leaf tissue using Nucleon Phytopure (from Amersham). Then, 10 μg of the extracted DNA was digested with HindIII, subjected to electrophoresis (the electrophoresis apparatus Electro-4 from Hybaid; 0.6% agarose gel (8.5×12 cm); phoresis at 25 V overnight (for 16 to 24 hrs)), transferred to Nylon Membrane Positive Charge (from Roche) by a capillary transfer method, and fixed at 120° C. for 30 minutes. Subsequently, a probe for detecting the HuCII×4 peptide was prepared using DIG Labeling & Detection Kit from Roche. Specifically, 10×DIG dNTP Labeling Mixture (from Roche), 1 unit of Ex Taq Polymerase (from Takara Bio Inc.), 10× Ex Taq Buffer, 0.4 μM each of the forward and reverse primers (SEQ ID NOS: 5 and 6) used in Example 1, and the plasmid DNA GluA-C4 (20 ng/μL) were mixed to a total volume of 20 μL. The mixture was subjected to PCR amplification for 35 cycles of 2 min. at 96° C., (30 sec. at 94° C., 1 min. at 62° C. and 2 min. at 72° C.) using Mastercycler (from Eppendorf) to prepare a DIG-labeled probe for detecting the CuHII×4 peptide. After one hour of prehybridization, the resultant probe was subjected to hybridization (2×SSC, 0.1% SDS, 68° C., 15 min.×twice) at 68° C. over a period of one night, washed, and then detected using Hyperfilm ECL (from Amersham).

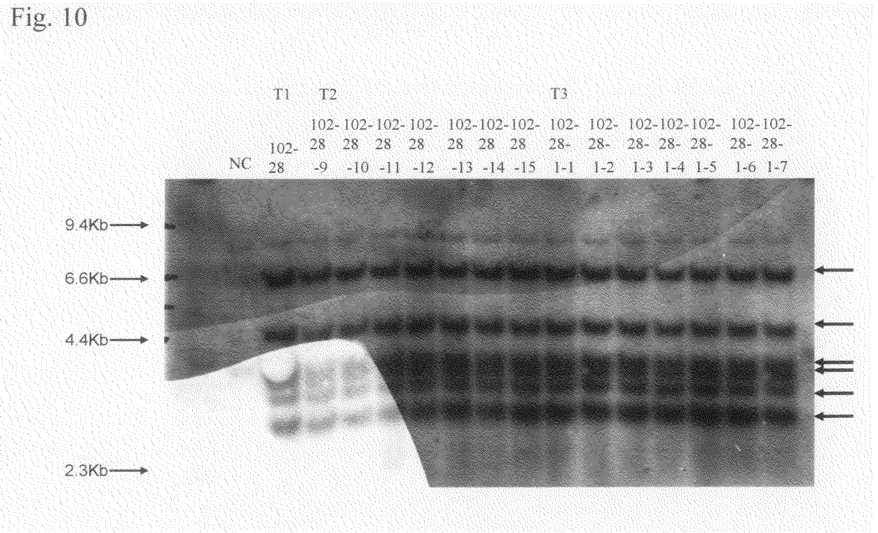

The results are shown in FIG. 10. As a result, it was demonstrated that the intended gene was introduced in pentaplicate or hexaplicate in the ultrahigh expression line and they were stably inherited as one factor to progenies thereof. These results suggested that the 5 to 6 copies of the intended gene were stably inherited as one factor to the progenies because the gene was collectively inserted into a narrow region (one gene locus) of one chromosome in the line.

As described above, it has been shown that the increased number of the copies of the peptide-encoding gene ligated together reduces the proportion of lines of a type separated from the drug marker but the introduction of the gene having the repeated sequences enables the breeding of a high expression (multicopy) line genetically stable over generations and free of the drug marker.

### Example 6

### Mouse Model Experiment on Autoimmune Response to Type II Collagen (Oral

### Immunotolerance Induction Experiment)

DBA/1 mice were immunized with purified bovine type II collagen and an adjuvant (FCA) to analyze fluctuations in the level of a collagen-specific IgG antibody in the serum by ELISA. Serum antibody response to the type II collagen and mild arthritis in the extremities were observed in all of the mice immunized; swelling with inflammation, in some of the mice. This demonstrated that experimental arthritis could be induced in the conditions used.

Using the mouse model experimental system, a positive control experiment was performed about immunotolerance induction by the oral administration of a glutelin-HuCII fusion protein-introduced rice to evaluate the effect thereof.

### 1. Method

### (1) Mice

Twenty-four DBA/1J mice (9-week old, female, from Japan SLC Inc.) were used. Mice were divided into 2 groups of 8 mice each; the mice were discriminated by punching ears thereof. The mice were bred by allowing to freely ingest commercial special solid feed free of fish flour (CLEA diet No. 012 from Clea Japan, Inc.) so that immunotolerance to collagen was not induced by the ingestion of collagen contained in the fish flour.

### (2) Feed Used for Induction of Oral Immunotolerance

The semiquantitative analysis performed in Example 4 showed that the C4 rice (No. 808-55) contained 1 μg of HuCII. 250-270 per grain. The composition of feed is described below which was set such that a mouse could ingest 25 μg of HuCII. 250-270 per day, assuming that the mouse ingested 5 g of feed per day. The calculation was carried out by setting the weight of one grain of rice to 18 mg. 18 mg×25 grains=450 mg. 450 mg/5 g=9%

TABLE 3

| | Std diet | With Rice flour |
|---|---|---|
| Composition | (weight %) | (weight %) |
| Protein | 25 | 25 |
| Starch | 40.8 | 31.8 |
| Sugar | 20 | 20 |
| Corn oil | 5 | 5 |
| Cellulose | 4 | 4 |
| Mineral | 3.5 | 3.5 |
| Vitamins | 1 | 1 |
| Choline chloride | 0.7 | 0.7 |
| Rice | 0 | 9 |

The feed was prepared using two types of rice from a recombinant rice plant (C4 TG-Rice (No. 808-55)) and a wild-type parent line, Koshihikari. When the feed was given to the mice, it was made into dumplings by adding water to powdered feed so that the amount thereof eaten was easily known. The dumpling was prepared by adding 3 ml of water to 5 g of powdered feed. However, when the “feed containing no rice” was prepared, a bovine CII solution (25 μg/3 ml) was used in place of water.

### (3) Induction of Oral Immunotolerance

The oral administration was carried out for 2 weeks; the feed was given in the amount of 80 g per group (on average 20 g per mouse) for one week. In other words, 200 μg of HuCII. 250-270 was ingested for each mouse together with Koshihikari, and bovine CII for two weeks.

### (4) Immunization

Bovine CII was intraperitoneally administered as an antigen 1, 4, 7 and 10 days after the end of the oral administration. Bovine CII was used after adding NaOH to the acetic acid solution thereof for neutralization. The dose thereof employed was 10 μg/100 μl per mouse for each administration.

### (5) Collection of Blood and Preparation of Serum

About 100 μl of blood was collected from the tail vein at the day of the end of the administration (0th day) and the 11th and 21st day thereafter. The blood was allowed to stand at room temperature for about 30 minutes and then at 4° C. overnight. After removing the blood clot, the centrifugation was performed at 17,500×g for 15 minutes. The resultant supernatant was collected to make a serum. The serum was stored at −20° C.

### (6) Measurement of Antibody Titer by ELISA Method

ELISA was performed by the following procedure according to an ordinary method. The ELISA plate was coated with a 10 μg/ml bovine CII solution. The mouse serum diluted by 1/100 with a 1% BSA/PBS-Tween was used as a primary antibody. As secondary antibodies, there were used POD-conjugated goat anti-mouse IgG (from Cell Signaling Technology), POD-conjugated goat anti-mouse IgG1 and rabbit anti-mouse IgG2a that were diluted by 1/10,000 with a 1% BSA/PBS-Tween. When the rabbit anti-mouse IgG2a was used, there was employed, as a tertiary antibody, POD-conjugated goat anti-rabbit IgG (from Cell Signaling Technology) diluted by 1/10,000 with a 1% BSA/PBS-Tween. The coloring time was 45 minutes.

### 2. Results

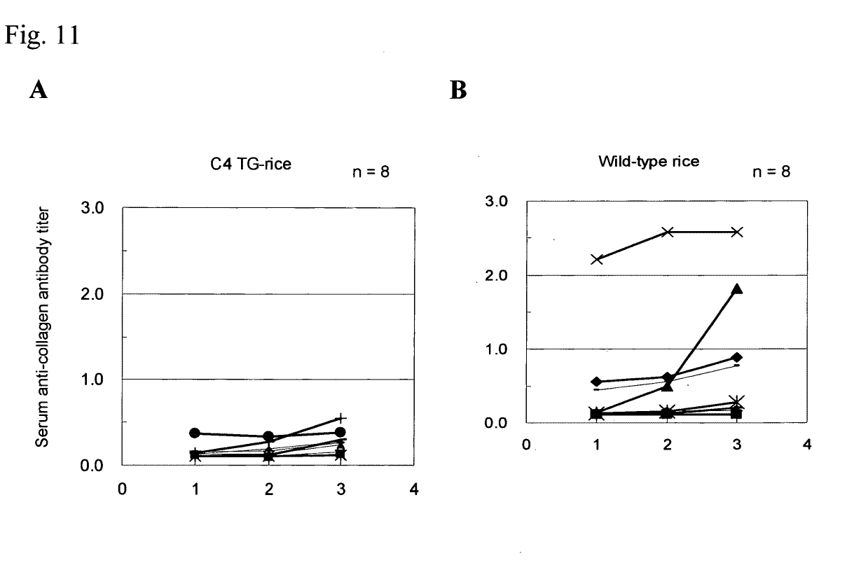

The results are shown in FIG. 11. As is apparent from FIG. 11, some mice ingesting the wild-type parent line rice had a high serum anti-collagen titer, while mice ingesting the recombinant rice had an extremely low serum anti-collagen titer at each point in time. This demonstrated that the ingestion of the recombinant rice induced immunotolerance in mice.

All publications, patents, and patent applications cited in this application are intended to be incorporated herein by reference in their entirety.

### INDUSTRIAL APPLICABILITY

According to the present invention, multiple copies of a gene can be introduced into a single gene locus, which permits the easy production of a multicopy line which is genetically stable and shows high expression. This enables a low-molecular peptide to be expressed and accumulated with a high efficiency in plants, particularly in plant seeds. Thus, the present invention is useful for the development of a new recombinant crop having the enhanced physiological functionality.

### Sequence Listing Free Text

SEQ ID NO: 1: Rice plant-derived GluA cDNA (inserted between the BamHI (5′) and EcoRI (3′) sites of pBluescript KS)
SEQ ID NO: 2: Rice plant-derived GluA
SEQ ID NO: 3: Epitope region peptide of human type II collagen (HuCII)
SEQ ID NO: 4: Codon-optimized HuCII base sequence
SEQ ID NO: 5: Description of artificial sequence: a primer for HuCII amplification (forward)
SEQ ID NO: 6: Description of artificial sequence: a primer for HuCII amplification (reverse)
SEQ ID NO: 7: GluPF2 promoter (inserted between the BamHI (5′) and EcoRI (3′) sites of pBluescript KS)
SEQ ID NO: 8: Description of artificial sequence: a primer for HuCII detection (forward)
SEQ ID NO: 9: Description of artificial sequence: a primer for HuCII detection (reverse)

1. A fusion protein expression vector comprising a gene encoding a member of the glutelin multigene family and two or more copies of a gene encoding an intended peptide composed of 3 to 40 amino acid residues ligated downstream of the gene under the control of a promoter, wherein the two or more copies of the gene can be multiply introduced, by multiple infection, into a single gene locus.

2. The vector according to claim 1, wherein the member of the glutelin multigene family is glutelin A or glutelin B.

3. The vector according to claim 1, wherein the promoter is the glutelin promoter.

4. The vector according to claim 1, wherein the fusion protein has peptide ligation sites consisting each of tyrosine or phenylalanine.

5. The vector according to claim 1, wherein the intended peptide is an epitope peptide of type II collagen.

6. The vector according to claim 1, wherein the vector is a binary-type hybrid vector comprising two T-DNA regions, wherein the first T-DNA region comprises two or more copies of the gene encoding the intended peptide composed of 3 to 40 amino acid residues ligated to the gene encoding the member of the glutelin multigene family and the second T-DNA comprises a selection marker.

7. A recombinant plant transformed with the vector according to claim 1.

8. The recombinant plant according to claim 7, wherein the plant is a rice plant.

9. The recombinant plant according to claim 8, wherein the rice plant is the rice variety “Koshihikari”.

10. The recombinant plant according to claim 7, wherein the two or more copies of the gene encoding the intended peptide are multiply introduced into a single gene locus.

11. (canceled)

12. A method for expressing and accumulating an intended peptide in plant seeds, comprising transforming the plant with the vector according to claim 1 and expressing the vector in seeds of the plant.

13. The method according to claim 12, further comprising the step of selecting, by DNA analysis, a plant free of selection marker from selfed progenies of the transformed plant.

14. (canceled)


SEQUENCE LISTING (9 sequences)

SEQ ID NO: 1 - DNA, 1800 bp, Artificial Sequence
  GluA cDNA derived from Oryza sativa (inserted between BamHI (5′) site and EcoRI (3′) site in pBluescript KS)
attggactccccgcggtggcggccgctctagaactagtggatccccctgcatccgccgtggcgcttgaggatgcaaagtacgacgaaaattcattagtactacaacaacatggcatccataaatcgccccatagttttcttcacagtttgcttgttcctcttgtgcaatggctctctagcccagcagctattaggccagagcactagtcaatggcagagttctcgtcgtggaagtccaagagaatgcaggttcgataggttgcaagcatttgagccaattcggagtgtgaggtctcaagctggcacaactgagttcttcgatgtctctaatgagcaatttcaatgtaccggagtatctgttgtccgtcgagttattgaacctagaggccttctactaccccattacactaatggtgcatctctagtatatatcatccaagggagaggtataacagggccaactttcccaggctgtcctgagtcctaccaacaacagttccaacaatcaggccaagcccaattgaccgaaagtcaaagccaaagtcaaaagttcaaggatgaacatcaaaagatccaccgtttcagacaaggagatgtaattgcattgcctgctggtgtagctcattggtgctacaatgatggtgaagtgccagttgttgccatatatgtcactgatctcaacaacggtgctaatcaacttgaccctaggcaaagggatttcttgttagctggaaataagagaaaccctcaagcatacaggcgtgaggttgaggagcggtcacagaacatatttagtggctttagcactgaactacttagcgaggctcttggcgtaagcggccaagtggcaaggcagctccaatgtcaaaatgaccaaagaggagaaattgtccgtgtcgaacacgggctcagtttgctgcagccatatgcatcattgcaggagcaggaacaaggacaagtgcaatcaagagagcgttatcaagaaggacaatatcagcaaagtcaatatggaagtggctgctctaacggtttggatgagaccttttgcaccctgagggtaaggcaaaacatcgataatcctaaccgtgctgatacatacaatccaagagctggaagggttacaaatctcaacacccagaatttccccattctcagtcttgtacagatgagtgcagtcaaagtaaatctataccagaatgcactcctttcaccattttggaacatcaacgctcacagcgtcgtgtatattactcaaggccgtgcccgggttcaagttgtcaacaccaatggaaagacagtgttcaacggcgagcttcgccgcggacagctgcttattataccacaacactatgcagttgtaaagaaggcacaaagagaaggatgtgcttacattgcattcaagaccaatcctaactctatggtaagccacattgcaggaaagagttccatcttccgtgctctcccaaatgatgttctagcaaatgcatatcgcatctcaagagaagaggctcagaggctcaagcataatagaggagatgagttcggtgcattcactccaatccaatacaagagctaccaagacgtttataatgcggcagaatcctcttaggtcggcttgcggataaagaataactaaataaataaattgcaagcaattgttttgctgctatgtactgtccagtctttcgactaatgatgataaagcctctctttatccttaaaaaaaaaaaaaaaaaaaaaggaattcgatatcaagcttatcgataccgtcgacctcgagggggggcccggtaccagctt

SEQ ID NO: 2 - protein, 499 aa, Artificial Sequence
  GluA derived from Oryza sativa (inserted between BamHI (5′) site and EcoRI (3′) site in pBluescript KS)
MASINRPIVFFTVCLFLLCNGSLAQQLLGQSTSQWQSSRRGSPRECRFDRLQAFEPIRSVRSQAGTTEFFDVSNEQFQCTGVSVVRRVIEPRGLLLPHYTNGASLVYIIQGRGITGPTFPGCPESYQQQFQQSGQAQLTESQSQSQKFKDEHQKIHRFRQGDVIALPAGVAHWCYNDGEVPVVAIYVTDLNNGANQLDPRQRDFLLAGNKRNPQAYRREVEERSQNIFSGFSTELLSEALGVSGQVARQLQCQNDQRGEIVRVEHGLSLLQPYASLQEQEQGQVQSRERYQEGQYQQSQYGSGCSNGLDETFCTLRVRQNIDNPNRADTYNPRAGRVTNLNTQNFPILSLVQMSAVKVNLYQNALLSPFWNINAHSVVYITQGRARVQVVNTNGKTVFNGELRRGQLLIIPQHYAVVKKAQREGCAYIAFKTNPNSMVSHIAGKSSIFRALPNDVLANAYRISREEAQRLKHNRGDEFGAFTPIQYKSYQDVYNAAESS

SEQ ID NO: 3 - protein, 21 aa, Artificial Sequence
  Description of Artificial Sequence human type II collagen epitope peptide(HuCII)
GPKGQTGKPGIAGFKGEQGPK

SEQ ID NO: 4 - DNA, 50 bp, Artificial Sequence
  Description of Artificial Sequence codon optimized nucleotide sequence enocoding human type II collagen epitope peptide(HuCII)
atgtcgacggcccaaagggccagccggcaagccaggcatcgccggcttca

SEQ ID NO: 5 - DNA, 51 bp, Artificial Sequence
  Description of Artificial Sequence primer for amplifying HuCII (Forward)
atgtcgacggcccaaagggccagaccggcaagccaggcatcgccggcttca

SEQ ID NO: 6 - DNA, 51 bp, Artificial Sequence
  Description of Artificial Sequence primer for amplifying HuCII (Reverse)
atctcgagatactttgggccctgctcgcccttgaagccggcgatgcctggc

SEQ ID NO: 7 - DNA, 842 bp, Artificial Sequence
  GluPF2 promoter (inserted between BamHI (5′) site and EcoRI (3′) site in pBluescript KS)
aagcttgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtactctaga

SEQ ID NO: 8 - DNA, 25 bp, Artificial Sequence
  Description of Artificial Sequence primer for detecting HuCII (Forward)
ctcagaggctcaagcataatagagg

SEQ ID NO: 9 - DNA, 25 bp, Artificial Sequence
  Description of Artificial Sequence primer for detecting HuCII (Reverse)
gagctcctactcgagatactttggg